(19) United States
(12) Patent Application Publication
(10) Pub. No.: US 2024/0090964 A1
(43) Pub. Date: Mar. 21, 2024

(54) AN APPARATUS CONFIGURED TO DRIVE AN OBJECT IN ONE LUMEN, VIA A DRIVER IN ANOTHER LUMEN

(71) Applicant: BIONAUT LABS LTD., Herzliya (IL)

(72) Inventors: Florent CROS, Los Angeles, CA (US); Alex KISELYOV, San Diego, CA (US); Suehyun CHO, Los Angeles, CA (US); Michael KARDOSH, Kiryat Ono (IL); Michael SHPIGELMACHER, Los Angeles, CA (US); Be'eri Berl KATZNELSON, Kiryat Tivon (IL)

(73) Assignee: BIONAUT LABS LTD., Herzliya (IL)

(21) Appl. No.: 18/255,509

(22) PCT Filed: Nov. 30, 2021
(86) PCT No.: PCT/US21/61149

§ 371 Date: Jun. 1, 2023

Related U.S. Application Data
(60) Provisional application No. 63/120,529, filed on Dec. 2, 2020.

(51) Int. Cl. A61B 34/00; A61B 10/02; A61B 90/00

(52) U.S. Cl. CPC A61B 34/73; A61B 10/0233; A61B 34/76; A61B 90/37; A61B 2090/3782

(57) ABSTRACT

An apparatus and methods of use thereof, the apparatus comprising: a medical device, configured to reside and to be manipulated within a first lumen; and a driving system, configured to manipulate—and/or control—the medical device, via a second lumen. The driving system comprising: a catheter, configured to be inserted into—and to navigate—or to be navigated—within the second lumen; and a driver, configured to travel within the catheter, and to remotely manipulate—and/or control—the medical device residing in the first lumen.

### BACKGROUND OF THE INVENTION

Accessing specific areas along the spine, to stimulate, to operate (ex: cut, distend, ablate, remove, etc.), or to a inject a substance for diagnostic or therapeutic reason, can be done in several ways. Direct puncture using a hypodermic needle is one such method. It does carry some risks.

Intrathecal catheterization (a catheter placed under the dura mater) offers another option. A long intrathecal catheter can be inserted around the lumbar region, a low risk location, and advanced to the site of interest, as high as the cervical area, for example.

Intrathecal catheters suffer from several limitations. One such limitation pertains to its maximum outer diameter. While a larger catheter can incorporate features to assist with bendable tips and guiding mechanism, these might be too large to safely advance under the dura. Another drawback comes from the overall length of the catheter. The longer the catheter, the more difficult it is to guide the distal end using torsion from the proximal end.

In contrast, Epidural catheterization is seen as relatively safe, even when using larger size catheter traveling outside the dura mater. Access can still be performed safely in the lumbar area and the tip of the catheter can be pushed and steered all the way to the cervical area using a gamut of mechanisms only available in larger lumen catheters.

Epidural catheter primary drawback is that it does not grant direct access to nerve roots. In the field of pain management for example, it is sometimes desirable to deliver a drug or a therapeutic substance directly at a particular nerve root location, under the dura mater. A direct exposure to the drug can circumvent the delayed response through the dura mater and possibly enhance or hasten the drug effects on the nerve.

There is therefore a need for an a device that can be both under and above the dura matter. Under the dura matter is the space targeted for the strategic injection of a drug, for example. The region above the dura can be used to assist with that task.

Therefore, there is a need for a sub-dural device that is substantially smaller than a typical intrathecal catheter that can be controlled and guided with greater precision.

### SUMMARY OF THE INVENTION

According to some embodiments of the invention, a new apparatus is provided, the apparatus comprising:
a medical device, configured to reside and to be manipulated within a first lumen; and
a driving system, configured to manipulate—and/or control—the medical device, via a second lumen; the driving system comprising:
   a catheter, configured to be inserted into—and to navigate—or to be navigated—within the second lumen; and
   a driver, configured to travel within the catheter, and to remotely manipulate—and/or control—the medical device residing in the first lumen.

According to some embodiments, the driver and the medical device are configured for magnetic communication between one and the other; and wherein:
the driver and the medical device comprise magnetic components; and/or
the driver comprises an electronic device, configured to produce a magnetic field.

According to some embodiments, the driver is configured to be manipulated within the catheter, by at least one of the following:
at least partially stiff guiding wire;
a remote magnetic field; and
any combination thereof.

According to some embodiments, the catheter is equipped with a mechanism a mechanical mechanism and/or an electro-mechanical mechanism, configured to allow the user to position the distal part of the catheter with precision.

According to some embodiments, the medical device comprises at least one of the following:
at least one chamber configured to accommodate and optionally release and/or load at least one substance into—and/or out of—the first lumen;
a sharp needle;
a blunt needle;
a blade;
a reflecting material for imaging purposes;
a magnetic material;
a dissecting tool;
a piercing tool;
at least one light emitting diode, for example a laser diode;
a coating configured to react to an electromagnetic stimulus;
at least one antenna configured to react to an electromagnetic stimulus;
at least one light sensor;
at least one ultrasound sensor;
at least one vital measuring sensor;
a set of integrated or discrete electronic components configured to harvest and store energy from an external radiation source, including but not limited to RF source;
at least one processor; and
and any combination thereof.

According to some embodiments, the driver comprises at least one of the following:
at least one magnet;
at least one magnetic field producer;
at least one light emitting diode (LED);
at least one laser diode;
at least one ultrasound source and/or sensor;
at least one vital measuring sensor;
at least one optic fiber for imaging purposes;
at least one optic fiber for energy and/or data transmission purposes;
a set of electrical leads to carry a current and/or send signals;
at least one processor; and
any combination thereof.

According to some embodiments, the control and the manipulation of the medical device are configured to perform at least one of:
deliver the medical device to a predetermined location, via at least one of: a dragging, pull, push and rotation and any combination thereof;
deliver at least one substance temporarily accommodated in the medical device, via remote communication with the medical device, optionally in a controllable method;
blunt dissection of tissue;
perforation of tissue;
ablation of tissues;
collection of at least one tissue to be accommodated with in the medical device;
tissue imaging;
tissue sensing using, for example, methods from the field of spectroscopy; and
any combination thereof.

According to some embodiments, the apparatus further comprising an introducer sheath, configured to allow the introduction- and optionally retrieval—of the medical device into—and optionally out of—the first lumen.

According to some embodiments, the apparatus further comprising a retrieving device, configured to retrieve the medical device out of the first lumen, via the introducer sheath.

According to some embodiments, the retrieving device comprises:
a pulling mechanism, located out of the first lumen; and
at least partially flexible retrieving wire, configured to reside in the first lumen; the retrieving wire's distal end is configured to communicate with the medical device, while the retrieving wire's proximal end is connected to the pulling mechanism.

According to some embodiments, the retrieving wire's distal end is:
connected to the medical device, such that it is configured to pull the medical device out the first lumen; or
in magnetic communication with the medical device, such that it is configured manipulate the medical device out of the first lumen.

According to some embodiments, the first lumen is the intrathecal space and the second lumen is the epidural space.

According to some embodiments, the introducer sheath is a lumbar sheath.

According to some embodiments of the invention, a new method is provided, for using the apparatus according to any one of the above mentioned embodiments; the method comprises:
inserting the catheter into the second lumen, and steering its distal end to a predetermined location, which enables a pre-selected procedure;
inserting the medical device into the first lumen and inserting the driver into the catheter, optionally in a simultaneous manner;
remotely manipulating and/or controlling the medical device, within the first lumen, via the driver.

According to some embodiments, the insertion of the medical device into the first lumen is provided via an introducer sheath.

According to some embodiments, the method further comprising manipulating the medical device, via the driver, to perform at least one of:
delivering the medical device to a predetermined location;
triggering the medical device to dispose at least one substance accommodated therewithin, at predetermined location and/or at a predetermined method;
dissecting tissue within the first lumen;
piercing tissue within the first lumen;
collecting tissue from the first lumen;
imaging and/or sensing tissue within the first lumen; and
any combination thereof.

According to some embodiments, the method further comprising scanning the medical device and/or a substance—accommodated therewithin or—disposed therefrom; wherein the scanning is provided by at least one selected from: ultrasound, X-ray, CT, MRI, thermal imaging and any combination thereof.

According to some embodiments, the scanning is provided by the driver.

According to some embodiments, the method further comprising retrieving the medical device out of the first lumen; and/or retrieving the driving system out of the second lumen.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically demonstrates an apparatus comprising a miniature medical device configured to manipulated and controlled in one lumen, via a driving system residing in an adjacent lumen, according to some embodiments of the invention: and

FIG. 2 schematically demonstrates another example of the apparatus.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

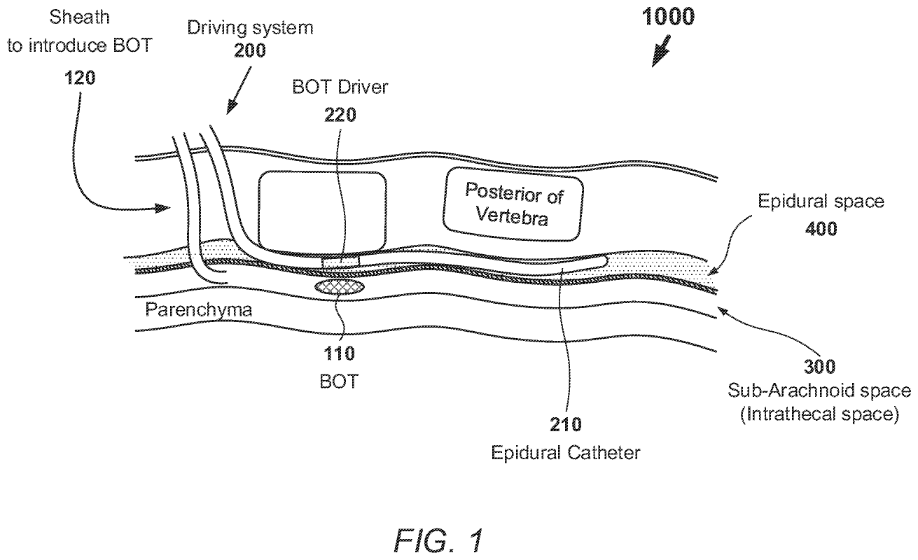

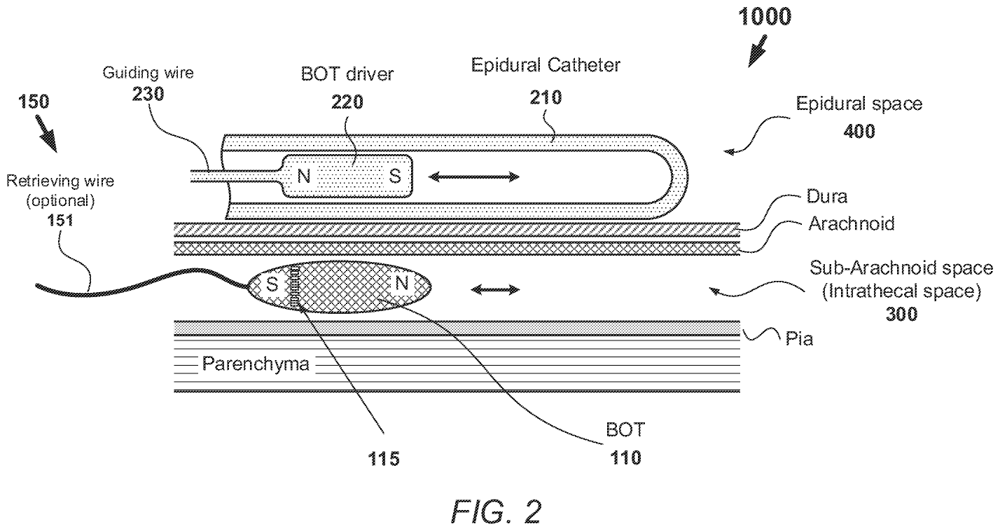

According to some embodiments of the invention, and as demonstrated in FIG. 1 and FIG. 2, an apparatus 1000 is provided, comprising:
a miniature medical device (e.g. a miniature robot and/or “bot”) 110, configured to reside and to be manipulated within a first lumen;
a driving system 200, configured to manipulate—and/or control—the medical device, via a second lumen; the driving system comprising:
   a catheter 210, configured to be inserted into—and to navigate- or to be navigated-within the second lumen 400;
   a driver 220, configured to travel within the catheter, and to remotely manipulate—and/or control—the medical device residing in the first lumen.

According to some embodiments, the term “manipulate”, may refer to at least one of: move, drag, pull, push, rotate, drive, and any combination thereof. According to some embodiments, the term “control”, may refer to at least one of: activate, trigger, provide instruction, communicate with, and any combination thereof.

According to some embodiments, the driver and the medical device are configured for magnetic communication therebetween; and wherein:
the driver and the medical device at least partially comprise magnetic components; and/or
the driver comprises an electronic device, configured to produce a magnetic field.

According to some embodiments, the magnetic communication is configured for the manipulation of the medical device. According to some embodiments the magnetic communication is configured for the control of the medical device. According to some embodiments the magnetic communication is configured for the data transformation between the medical device and the driver and vice versa.

According to some embodiments, the driver and the medical device are configured for light communication therebetween, for example via at least one of: LED/laser diode, sensor/s, processor/s and any combination thereof. According to some embodiments, the light communication is configured for the control of the medical device. According to some embodiments, the light communication is configured for the data transformation between the medical device and the driver. According to some embodiments, a light and/or a laser beam provided by the driver via the second and first lumens, can be received by the medical device, or vise versa. For example, the light/laser beam from the driver can induce dissolution of a substance around and/or within the medical device.

According to some embodiments, the driver 220 is configured to be manipulated within the catheter 210, by at least one of:
at least partially stiff guiding wire 230, threaded within the catheter; for example, in communication with the driver via its distal end, and controlled via the proximal edge of the catheter;
a remote magnetic field (not shown); and
any combination thereof.

According to some embodiments, the medical device (e.g. the miniature robot) comprises at least one of:
at least one chamber 115 configured to accommodate and optionally release and/or load at least one substance into—and/or out of—the first lumen;
a sharp needle;
a blunt needle;
a blade;
a reflecting material, for imaging purposes;
a magnetic material;
a dissecting tool;
a piercing tool;
at least one LED and/or laser diode;
at least one light and/or laser sensor;
at least one ultrasound sensor;
at least one vital measuring sensor;
at least one processor; and
any combination thereof.

According to some embodiments, the driver comprises at least one of:
at least one magnet and/or magnetic material;
a magnetic field producer;
at least one LED and/or laser diode;
at least one light and/or laser sensor;
at least one ultrasound sensor;
at least one vital measuring sensor;
at least one processor; and
any combination thereof.

According to some embodiments, the control and the manipulation of the medical device are configured to perform at least one of:
deliver the medical device to a predetermined location, via at least one of: dragging, pulling, pushing and rotation thereof;
deliver and dispose at least one substance temporarily accommodated in the medical device, via remote communication with the medical device, optionally in a controllable method; for example, control of at least one of: dosage, rate, duration, location, path and any combination thereof;
blunt dissection of tissue; for example tissue surrounding, tumor, blood clot and more;
perforation of tissue; for example tissue surrounding, tumor, blood clot and more; collection of at least one tissue to be accommodated with in the medical device; for example, specimen/s collection;
tissue imaging; for example, via ultrasound, or by providing a reflecting material to be remotely imaged; and any combination thereof.

According to some embodiments, the apparatus further comprising an introducer sheath 120, configured to allow the introduction—and optionally the retrieval—of the medical device 110 into—and optionally out of—the first lumen 300.

According to some embodiments, the driver is further configured to manipulate the medical device out of the first lumen, and optionally out of the body.

According to some embodiments, the apparatus further comprising a retrieving device 150, configured to retrieve the medical device 110 out of the first lumen 300, via the introducer sheath 120. According to some embodiments, the retrieving device comprises:
a pulling mechanism (not shown) located out of the first lumen;
at least partially flexible retrieving wire 151, configured to reside in the first lumen; the retrieving wire's distal end is configured to communicate with the medical device, while the retrieving wire's proximal end is connected to the pulling mechanism.

According to some embodiments, the retrieving wire's distal end is:
connected to the medical device, such that it is configured to pull the medical device out the first lumen; or
in magnetic communication with the medical device, such that it is configured manipulate the medical device out of the first lumen.

According to some embodiments, the first lumen and the second lumen are adjacent one to the other. According to some embodiments, the first lumen and the second lumen may at least partially share a wall, a tissue membrane.

According to some embodiments, the first lumen in at least some of the above mentioned embodiments is the intrathecal space and the second lumen in at least some of the above mentioned embodiments is the epidural space. According to some embodiments, the introducer sheath is a lumbar sheath.

According to some embodiments of the invention, a method is provided, for using the apparatus 1000 according to any one of the above mentioned embodiments. The method comprises:
inserting the catheter 210 into the second lumen 400, and steering its distal end to a predetermined location, which enables a pre-selected procedure;
inserting the miniature medical device 110 into the first lumen 300 and inserting the driver 220 into the catheter 210, optionally in a simultaneous manner;
remotely manipulating and/or controlling the medical device, within the first lumen, via the driver.

According to some embodiments, the insertion of the medical device into the first lumen is provided via an introducer sheath 120.

According to some embodiments, the method further comprising manipulating and/or controlling the medical device, via the driver, to perform at least one of:
delivering the medical device to a predetermined location, via at least one of: dragging, pulling, pushing and rotation thereof;
delivering and disposing at least one substance temporarily accommodated in the medical device, via remote communication with the medical device, optionally in a controllable method; for example, control of at least one of: dosage, rate, duration, location, path and any combination thereof;
blunt dissecting of tissue; for example tissue surrounding, tumor, blood clot and more;
perforating of tissue; for example tissue surrounding, tumor, blood clot and more; collecting at least one tissue to be accommodated with in the medical device; for example, specimen/s collection;
tissue imaging; for example, via ultrasound, or by providing a reflecting material to be remotely imaged; and any combination thereof.

According to some embodiments, the method further comprising scanning the medical device and/or at least one substance—accommodated therewithin or—disposed therefrom; wherein the scanning is provided by at least one selected from: ultrasound, X-ray, CT, MRI, thermal imaging and any combination thereof.

According to some embodiments the scanning is provided by the driver.

According to some embodiments, the method further comprising retrieving the medical device out of the first lumen. According to some embodiments, the method further comprising retrieving the driving system out of the second lumen.

According to some embodiments of the invention, an apparatus is provided that can bridge drawbacks disclosed in the Background chapter. The apparatus comprising:
an Epidural Catheter (EC) configured to house a driver and that can be steered safely, within the EC to a site of interest;
a Lumbar Sheath (LSh) system that can be introduced temporarily into the intrathecal space;
an untethered object, for example a miniature robot, that can be introduced into the intrathecal space, using the LSh; and
a tethered—or an untethered—driver, that can travel inside the EC, and remotely manipulate and/or control the miniature robot residing in the intrathecal space.

According to some embodiments, the apparatus further comprising a mechanical device, configured to latch onto the robot and assist with retrieving the robot out of the first lumen and optionally out of the body.

According to some embodiments, the Epidural Catheter (EC) is configured to house the driver, which can be safely steered to the site of interest, in the epidural space.

According to some embodiments, the EC is designed to navigate and reside in the epidural space. According to some embodiments, the primary function of the EC is to provide a corridor for the driver to travel along the spine, without directly being in contact with the surrounding tissues. According to some embodiments, the EC is essentially introduced only once and, assuming that the tip of the EC reaches its intended destination, no further motion of the EC is to be expected. It is the driver, inside the EC, that is configured to travel several times along the spine.

According to some embodiments, it is not expected that the catheter shall remain in the epidural space indefinitely, but should rather reside for the duration of the use of the apparatus. The epidural space is a relatively safe space around the spinal cord.

According to some embodiments, the EC inner and outer diameter, material, durometer, length, radiopacity and other attributes are chosen to at least one of:
ease the travel in the Epidural Space (EDS), under fluoroscopy or other imaging means; and
allow a driver, of adequate size, to travel along the entirety or specific section of the EC.

According to some embodiments, the EC can be built with specific features that ease the steering of its distal tip.

According to some embodiments, the EC proximal end can comprise a handle with various degrees of complexity, configured to enable the manipulation of the EC within the Epidural space.

According to some embodiments, the EC handle is configured to help the operator to access a variety of functions, selected from:
steering the catheter;
moving the driver along the catheter;
triggering certain electric, electronic or mechanical functions that might be built within the driver; and
any combination thereof.

According to some embodiments, a sub-Dural Lumbar Sheath (LSh) can be introduced temporarily into the intrathecal space.

According to some embodiments, LSh can be introduced in the lumbar area using a number of techniques, for example, one similar following the Seldinger Methodology. According to some embodiments, the primary function of the LSh is to provide a safe and reliable route to enter and exit the intrathecal space for a short duration of time. According to some embodiments, the LSh can remain in use for a few hours at the time before being removed and the puncture site is closed up.

According to some embodiments, the LSh inner and outer diameter, material, durometer (e.g. material hardness), length, radiopacity and other attributes are chosen to at least one of:
minimize the size of the puncture in the Dura mater;
maximize the size of the inner lumen to allow a medical robot of adequate size to travel in and out;
minimize the length of the sheath to limit possible damage to the cord;
ease the introduction of a mechanical device that can latch on the robot and assist with the retrieval of the robot; and
any combination thereof.

According to some embodiments, the LSh further comprises an access port, on the proximal end, configured to allow easy entry or retrieval of the robot.

According to some embodiments, the robot and the driver are configured to communicate therebetween.

According to some embodiments, the robot is located outside the EC, while the diver is located inside the EC. According to some embodiments, for specific applications, the robot can reside inside the sub-Dural space. In this case, the minimum distance that can, in theory, separate the robot from the driver is approximately the sum of the thickness of the EC wall and the thickness of the Dura matter. Both EC wall and Dura matter can be in the order of a few hundred of micrometer thick.

According to some embodiments, the primary function of the robot can vary. According to some embodiments, the robot can be used as a vessel to carry and release a drug in a controllable or predictable fashion. According to some embodiments, the robot can also be designed to perform an activity physical in nature, including but not limited to (a) blunt dissection, (b) dissection or (c) perforation.

According to some embodiments, the primary function of the driver is to induce the robot to move when the driver is pushed or pulled inside the EC lumen using a tether, for example According to some embodiments, the driver can be equipped with additional functions related to the robot (e.g.: help trigger the release of a drug) or not. According to some embodiments, the functions that are not related to the robot operation can include, for example, ultrasound mapping of the surrounding tissues, etc.

According to some embodiments, a variety of attributes can pertain to the robot and/or the driver, selected from:
the robot can comprise magnetic materials, such as a permanent magnet or a ferromagnetic metal;
the driver can also comprise a magnetic material so that predicable and reproducible magnetic forces between the two objects: robot and driver can be generated;
the robot can comprise a reservoir for a drug, and/or a release mechanism to release the drug;
the robot can also comprise a mechanical appendix, such as a retractable needle and/or blade;
the driver can comprise a plurality of magnets that can (1) magnify the forces between the BOT and the DRIVER and (2) be used to trigger a mechanism insider the robot, if necessary;
the driver can be tethered;
the tether of the driver can comprise electrical wires;
the driver can comprise, in addition to a magnet, one or more electric or electronic components; the Electric and electronic components can in include LEDs, laser diodes, acoustic or ultrasound sensors, and more;
the electric and electronic components can be used to trigger a mechanism located inside the robot;
the electric and electronic components can be used to measure local environmental variables or provide a means to visualize the surrounding tissues for therapeutic or diagnostic purposes; and any combination thereof.

According to some embodiments, the mechanical device that can latch on the robot can comprise a soft flexible wire, known as a guide wire comprising a magnet at the distal end. According to some embodiments, the magnet is chosen to preferentially latch on the robot. According to some embodiments, the latching occurs when the robot is brought within the vicinity of the distal end of the mechanical device for retrieval.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An apparatus comprising:
a medical device, configured to reside and to be manipulated within a first lumen; and
a driving system, configured to manipulate—and/or control—the medical device, via a second lumen; the driving system comprising:
a catheter, configured to be inserted into—and to navigate- or to be navigated-within the second lumen; and
a driver, configured to travel within the catheter, and to remotely manipulate—and/or control—the medical device residing in the first lumen.

2. The apparatus of claim 1, wherein the driver and the medical device are configured for magnetic communication therebetween; and wherein:
the driver and the medical device comprise magnetic components; and/or
the driver comprises an electronic device, configured to produce a magnetic field.

3. The apparatus of claim 1, wherein the driver is configured to be manipulated within the catheter, by at least one of:
at least partially stiff guiding wire;
a remote magnetic field; and
any combination thereof.

4. The apparatus of claim 1, wherein the medical device comprises at least one of:
at least one chamber configured to accommodate and optionally release and/or load at least one substance into—and/or out of—the first lumen;
a sharp needle;
a blunt needle;
a blade;
a reflecting material for imaging purposes;
a magnetic material;
a dissecting tool;
a piercing tool;
at least one LED and/or laser diode;
a coating configured to react to an electromagnetic stimulus;
at least one antenna configured to react to an electromagnetic stimulus;
at least one light sensor;
at least one ultrasound sensor;
at least one vital measuring sensor;
a set of integrated or discrete electronic components configured to harvest and store energy from an external radiation source;
at least one processor; and
and any combination thereof.

5. The apparatus of claim 1, wherein the driver comprises at least one of:
at least one magnet;
at least one magnetic field producer;
at least one LED;
at least one laser diode;
at least one ultrasound source and/or sensor;
at least one vital measuring sensor;
at least one optic fiber for imaging purposes;
at least one optic fiber for energy and/or data transmission purposes;
a set of electrical leads to carry a current and/or send signals;
at least one processor; and
any combination thereof.

6. The apparatus of claim 1, wherein the control and the manipulation of the medical device are configured to perform at least one of:
deliver the medical device to a predetermined location, via at least one of: a dragging, pull, push and rotation and any combination thereof;
deliver at least one substance temporarily accommodated in the medical device, via remote communication with the medical device, optionally in a controllable method;
blunt dissection of tissue;
perforation of tissue;
ablation of tissues;
collection of at least one tissue to be accommodated with in the medical device;
tissue imaging;
tissue sensing; and
any combination thereof.

7. The apparatus of claim 1, further comprising an introducer sheath, configured to allow the introduction—and optionally retrieval—of the medical device into—and optionally out of—the first lumen.

8. The apparatus of claim 7, further comprising a retrieving device, configured to retrieve the medical device out of the first lumen, via the introducer sheath.

9. The apparatus of claim 8, wherein the retrieving device comprises:
a pulling mechanism, located out of the first lumen;
at least partially flexible retrieving wire, configured to reside in the first lumen;
the retrieving wire's distal end is configured to communicate with the medical device, while the retrieving wire's proximal end is connected to the pulling mechanism.

10. The apparatus of claim 9, wherein the retrieving wire's distal end is:
connected to the medical device, such that it is configured to pull the medical device out the first lumen; or
in magnetic communication with the medical device, such that it is configured manipulate the medical device out of the first lumen.

11. The apparatus of claim 1, wherein the first lumen is the intrathecal space and the second lumen is the epidural space.

12. The apparatus of claims 7 and 11, wherein the introducer sheath is a lumbar sheath.

13. A method of using the apparatus according to any one of claims 1 to 12, the method comprises:
inserting the catheter into the second lumen, and steering its distal end to a predetermined location, which enables a pre-selected procedure;
inserting the medical device into the first lumen and inserting the driver into the catheter, optionally in a simultaneous manner;
remotely manipulating and/or controlling the medical device, within the first lumen, via the driver.

14. The method of claim 13, wherein the insertion of the medical device into the first lumen is provided via an introducer sheath.

15. The method of claim 13, further comprising manipulating the medical device, via the driver, to perform at least one of:
delivering the medical device to a predetermined location;
triggering the medical device to dispose at least one substance accommodated therewithin, at predetermined location and/or at a predetermined method;
dissecting tissue within the first lumen;
piercing tissue within the first lumen;
collecting tissue from the first lumen;
imaging and/or sensing tissue within the first lumen; and
any combination thereof.

16. The method of claim 13, further comprising scanning the medical device and/or a substance—accommodated therewithin or—disposed therefrom; wherein the scanning is provided by at least one selected from: ultrasound, X-ray, CT, MRI, thermal imaging and any combination thereof.

17. The method of claim 16, wherein scanning is provided by the driver.

18. The method of claim 13, further comprising retrieving the medical device out of the first lumen.
